(19) United States
(12) Patent Application Publication
(10) Pub. No.: US 2010/0009922 A1
(43) Pub. Date: Jan. 14, 2010

(54) HYPOTENSIVE PEPTIDES FROM SOY PROTEINS AND METHOD OF PREPARATION

(76) Inventors: Li Chen, Hong Kong (CN); Jian-Er Lin, Hong Kong (CN); Kee Hung Chu, Hong Kong (CN)

Correspondence Address: WILKINSON & GRIST, 6TH FLOOR, PRINCE''S BUILDING, CHATER ROAD, CENTRAL, HONG KONG, HK

(21) Appl. No.: 12/497,757

(22) Filed: Jul. 6, 2009

Related U.S. Application Data
(60) Provisional application No. 61/078,344, filed on Jul. 4, 2008.

(51) Int. Cl. A61K 38/08; C12P 21/00; A61K 38/10; C07K 7/06; C07K 7/08

(52) U.S. Cl. 514/14; 435/71.2; 514/15; 530/327; 530/326; 514/17; 514/16

(57) ABSTRACT

A method for preparing an anti-hypertension peptide composition, using a microbe which is intrinsic to common soybean food and capable of producing protease. The composition mainly contain peptides with a molecular weight of less than 10 kD which are produced by decomposition of soy proteins by the microbe in a fermentation process. The composition is shown to have an ACE inhibitory activity and an effect of lowering blood pressure when administered to a mammalian subject.

### CROSS REFERENCE TO RELATED APPLICATION

This application claims benefit from U.S. Provisional Application No. 61/078,344, filed on Jul. 4, 2008, the content of which is hereby incorporated by reference in its entirety.

### FIELD OF THE INVENTION

This invention relates to a peptide and a pharmaceutical composition for treating patients suffering from a high blood pressure. Particularly, it relates to a peptide obtained from soybean proteins by a process of fermentation.

### BACKGROUND OF THE INVENTION

Hypertension is one of the risk factors for strokes, heart attacks, and heart failure, and is a leading cause of chronic renal failure. Even moderate elevation of arterial blood pressure leads to shortened life expectancy. At severely high pressures, defined as mean arterial pressures 50% or more above average, a person can expect to live no more than a few years unless appropriately treated. The angiotensin I—converting enzyme (ACE) is physiologically important in the regulation of blood pressure. It catalyses the conversion of angiotensin I to a potent vasoconstrictor angiotensin II, as well as inactivating the vasodilating nonapeptide bradykinin. These two actions of ACE make it an ideal target in treating conditions such as high blood pressure, heart failure, diabetic nephropathy and type 2 diabetes mellitus [1]. Inhibition of ACE reduces the activity of angiotensin-II but increases bradykinin levels, and thus result in lowering of blood pressure [2].

Currently, hypertension is often treated with the use of anti-hypertensive pharmaceutical products including calcium channel blockers, beta blockers, diuretics, alpha blockers, angiotensin II antagonists and ACE inhibitors, such as Captopril and Enalapril [3]. While such pharmaceutical products are effective in decreasing elevated blood pressure, some of them often have side effects associated with their use.

Alternatively, several compounds produced from food stuffs have been studied for their ability to reduce blood pressure. These ACE inhibitory substances from natural sources such as milk, soybean protein or fish meat protein are proposed for practical use as antihypertensive agents, having low toxicity and great safety. Bioactive peptides derived from food commonly contain 2 to 9 amino acids [4]. However, this range may be extended to 20 or more amino acids units [5].

Soybean contains about 40% protein and thus has the potential to be an important source of bioactive peptides. The major components of soybean storage proteins are β-conglycinin and glycinin, which account for approximately 70% of total seed proteins [6]. Several studies suggested that soybean proteins and soybean-derived peptides may play an important role in disease prevention and treatment. Food processing and in vivo enzyme digestion of soy proteins may release peptides, which exert diverse and unique health benefits that can be used in the prevention of age-related chronic disorders such as cardiovascular disease, cancer, obesity, and decreased immunity. With the increasing knowledge in physiological activities of bioactive soy peptides, the commercial interest to use them as active ingredients in food and drug keep growing.

Chen and others [7] [8] identified the angiotensin I-converting enzyme inhibitory peptides in the peptic digest of soybean protein. Four active ACE inhibitory peptides were found, including: Ile-Ala, Tyr-Leu-Ala-Gly-Asn-Gln (SEQ ID NO. 11), Phe-Phe-Leu, and Ile-Tyr-Leu-Leu (SEQ ID NO. 12). Antihypertensive peptides were also found in soybean alcalase digest [9]. Kodera and Nio (2002) (US Patent 2004/0014670A1) have produced five peptides by soybean protein digestion with protease D3: (1) Tyr-Val-Val-Phe-Lys (SEQ ID NO. 13), (2) Pro-Asn-Asn-Lys-Pro-Phe-Gln (SEQ ID NO. 14), (3) Asn-Trp-Gly-Pro-Leu-Val (SEQ ID NO. 15), (4) Ile-Pro-Pro-Gly-Val-Pro-Tyr-Trp-Thr (SEQ ID NO. 16), (5) Thr-Pro-Arg-Val-Phe (SEQ ID NO. 17).

Fermentation may be considered to be an efficient way to produce ACE inhibitory peptides, since the bioactive peptides can be released by the enzyme derived from microorganisms. However, there remains a number of challenges for a fermentation process to be utilized to produce more effective and safer peptide products for treating hypertension: 1) selection of unique microbial strains for efficient production of peptides, 2) optimum control of the fermentation and downstream processes for peptide production and purification, 3) identification of the effective peptide chemistry, and 4) use of the peptides for treating hypertension. As a result, there is a need to develop a more effective fermentation method to the inventors' knowledge that can be used to obtain bioactive peptides for treating hypertension.

### SUMMARY OF THE INVENTION

One object of the present invention is to provide a method through which peptides with effective ACE inhibitory activity and useful for treating hypertension can be identified and produced from natural sources.

Another object of the present invention is to provide antihypertensive peptides, which exhibit effective ACE inhibitory activity and may be employed as a pharmaceutical, a dietary supplements or a food ingredient. Another object of the present invention is to provide methods with which these peptides can be produced and used for treating hypertension.

These and other objects have been realized by the present invention by providing a method which relies on the bacterial species which are intrinsic to the common soybean food to isolate suitable microbes and to carry out fermentation using the isolated benign microbes. It is surprisingly found that peptides having exceptionally high ACE inhibitory activity can be obtained by fermentation of soybean protein with Bacillus species commonly find in the soybean food. The released soy peptides by such fermentation are then partially purified by centrifugation and ultrafiltration, and conveniently administered to people for their beneficial effect on blood pressure. Furthermore, using the processes developed by this invention, novel peptides with unique chemistry and showing beneficial health effect on blood pressure can be identified and can be synthesized and suitable for large-scale production.

With either the partially purified peptide mixture or the purified or synthesized particular individual peptide, the invention provides a new method to prevent and treat hypertension.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF DRAWINGS

FIG. 1 shows the effect on SHR blood pressure (continuous administration) of the soy peptide mixture (CKPN001).

FIG. 2 shows the effect on SHR arterial pressure (single dose administration) of the soy peptide mixture (CKPN001).

FIG. 3 shows the result of mass spectrometric analysis of the soy peptides mixture (CKPN001), showing the peptide profile and molecular weight distribution. a): the left part of the spectrum; b): the right part of the spectrum.

### DETAILED DESCRIPTION OF PARTICULAR EMBODIMENTS

### Isolation of Protease Producing Microbes from Soy foods

Ten grams of natto, a type of common soybean food, were added to 30 ml distilled water, shaken vigorously and held at 75° C. for 20 minutes to destroy vegetative microbial cells. The suspension was then plated on nutrient agar (containing 3 g/L beef extract, 5 g/L peptone and 15 g/L Agar) and incubated at 30° C. for 24 hours. Bacterial colonies were isolated and characterized by their colony types, Gram staining, spore formation, and growth conditions. The isolated bacterial isolates were plated onto skim milk agar plates (containing 25 g/L skimmed milk, 1 g/L peptone, 5 g/L sodium chloride and 15 g/L Agar) and were incubated at 30° C. for 24 hours. A clear zone around the colonies on the skim milk agar plate indicated that the particular isolated colony could produce protease. Depending on the zone of clearance and growth temperature of the organism, a Bacillus species, which produces largest clearance zones with growth temperature at about 30° C. (as characterized in the preceding step) were selected for further experimental studies.

The same process was repeated with furu (a salty to fu), another type of common soybean food purchased from a supermarket and similar results were obtained. For the purpose of this invention, the term “soybean food” includes, but is not limited to, natto, douchi (a naturally fermented soy), furu, ground bean sauce, and bean paste. They can all provide the source of the protease-producing microbial species needed to practice the present invention.

After the fermention (as described in the following step), protease activity was determined according to the method of Rick, W. [10]. To 2.9 ml of substrate (0.8 mM Na-p-Tosyl-L-Arginine Methyl Ester in 100 mM Potassium Phosphate Buffer, pH 8.0), 0.1 ml of culture broth was added and immediately mixed by inversion and the increase in absorbance at 247 nm was measured. One unit of enzyme activity is defined as the amount of enzyme required to hydrolyze 1.0 μmole of Na-p-Tosyl-L-Arginine Methyl Ester per minute at pH 8.0 and 25° C. Protein concentration was determined by the method of Lowry et al., with bovine serum albumin (Sigma Chemical Company, St. Louis, Mo.) as the standard.

Preparation of Anti-hypertension Peptides from Soy Protein by Microbial Fermentation

Fifty grams of commercially available soy protein (manufactured by NANTONG Sun-Green Bio-Tech CO., LTD) and 50 grams of D-glucose were dissolved in 1 liter of water, respectively. After sterilization at 121° C. for 20 minutes, 350 ml soy protein solution was mixed with 150 ml D-glucose solution and the resultant mixture was cooled to 30° C. The mixture was inoculated with a primary starter containing ca. 2×10⁸ Bacillus cells of a species selected in the preceding step. The fermentation was then carried out at 30° C. for 48 hours in 1-liter shake flask. The decomposition of soy protein was analyzed by SDS-polyacrylamide gel electrophoresis and Lowry method of protein quantitation. This fermented soy protein mixture was centrifuged and ultra-filtered through ultra-filtration membrane with a molecular weight cutoff of 10 kD (manufactured by Millipore Corporation). The filtrate was freeze-dried, resulting in approximately 11 g of dry product, is referred to as “CKPN001” in this application. CKPN001 was the preparation of soy peptides used for all the tests disclosed in this application.

CKPN001 with the beneficial effects as described in the following can be produced with any Bacillus species which is intrinsic to common soybean food and isolated and selected by the process according to the present invention as detailed in the above.

### Separation of Peptides by HPLC

The partially purified soy peptide mixture (CKPN001) was fractionated by a reverse phase column Alltech C18 (250×22 mm). Solution A (distilled water containing 0.1% TFA) and solution B (acetonitrile containing 0.1% TFA) were used as the mobile phase, and the HPLC analysis was carried out using a concentration gradient technique with which the concentration of solution B increased from 0% to 25% for 48 mins at a flow rate of 10 ml/min. Peptides in each fraction was analyzed by a UV-detector. For each eluted fraction, the inhibition activity of angiotensin converting enzyme was determined by the method described in the following.

### Proteomics Analysis of Soy Peptides by Means of MALDI TOF/TOF Analyzer

Mass spectrometric analysis was carried out using a MALDI-TOF/TOF tandem mass spectrometer ABI 4700 proteomics analyzer (Applied Biosystems, Foster City, USA). For acquisition of mass spectra, 0.5 μL samples were spotted onto a MALDI plate, followed by 0.5 μL matrix solution (4 mg/ml α-cyano-4-hydroxycinnamic acid in 35% ACN and 1% TFA). Mass data acquisitions were piloted by 4000 Series Explorer™ Software v3.0 using batched-processing and automatic switching between MS and MS/MS modes. Fragmentation of precursor ions was performed using MS-MS 1 kV positive mode with CID on and argon as the collision gas. MS/MS spectra were accumulated from 3000 laser shots using default calibration with Glu-Fibrinopeptide B from 4700 Calibration Mixture (Applied Biosystems, USA). Data were processed and submitted for database searching with GPS Explorer™ software v3.5 (Applied Biosystems, Foster City, USA) and searched against NCBInr database 4914404 sequences (released on May 14, 2008) by Mascot search engine version 1.9.05 (Matrix science, London, UK).

Each of the RP-HPLC fractions was analyzed by MALDI TOF/TOF analyzer in order to determine their constituent peptides. As shown in Table 4, six peptides were identified from the active fractions of 24-1, 25-2, 26-1, 27-1, 27-2, 31-2. Except for peptide FNQRSPQ, all of the other five peptides were derived from the subunit of Glycinin.

| | | | | | |
|---|---|---|---|---|---|
| HPLC | | | | Ion | |
| Fraction No | Calc. Mass | Obsrv. Mass | Se\|uence | Score | Protein/gi |

| | | | | | | |
|---|---|---|---|---|---|---|
| #1 | 24-1 | 742.4206 | 742.4281 | LKPDNR | 23 | Glycinin G2 |
| | | | | | | Precursor/gi\|121277 |
| #2 | 25-2 | 1198.5811 | 1198.6213 | VNPESQQGSPR | 46 | Glycinin/gi\|4249568 |
| #3 | 26-1 | 766.3842 | 766.3996 | LEPDHR | 34 | Glycinin/gi\|4249568 |
| #4 | 27-1 | 876.4322 | 876.4581 | FNQRSPQ | 32 | Alpha subunit of beta |
| | | | | | | conglycinin/gi\|9967357 |
| #5 | 27-2 | 1243.6389 | 1243.6637 | LRSPDDERKQ | 31 | Glycinin/gi\|4249566 |
| #6 | 31-2 | 1209.6698 | 1209.7023 | LVPPKESQRR | 29 | Glycinin G2 |
| | | | | | | Precursor/gi\|121277 |

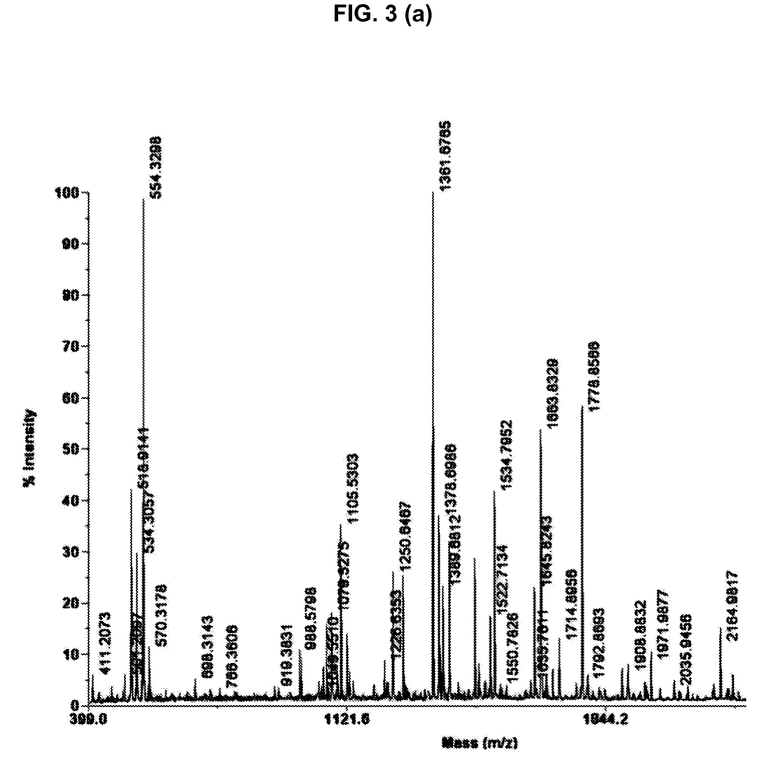

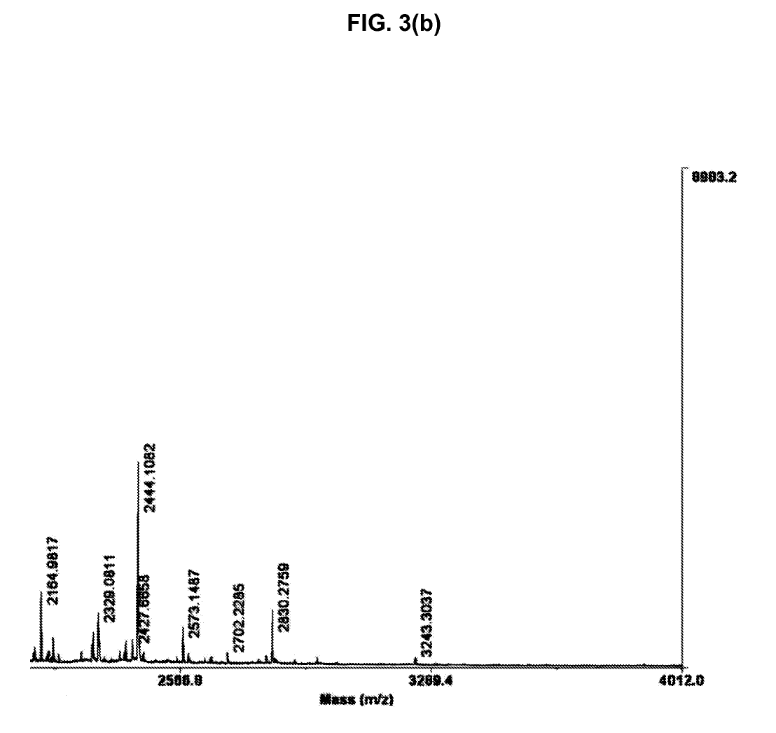

Peptides in CKPN001 before RP-HPLC separation were also analyzed by MALDI TOF/TOF analysis. Total 73 peptides from alpha subunit of beta conglycinin were identified via database searching. As shown in FIG. 3, peptides had relatively high intensity, including 1361.6747 (PFPRPPHQKEE: SEQ ID NO. 1), 1778.868 (DERPFPFPRPPHQK: SEQ ID NO. 2), 1663.838 (ERQFPFPRPPHQK: SEQ ID NO. 3), 1534.7952 (RQFPFPRPPHQK: SEQ ID NO. 4), 1378.6985 (QFPFPRPPHQK: SEQ ID NO. 5), and 1407.689 (ERQFPFPRPPH: SEQ ID NO. 6) were all derived from the alpha subunit of beta conglycinin region 87-102. Peptides f87-100, f88-100, f89-100, and f90-100 were all ended with the amino acid residue -Lys, suggesting the proteinase released from the Bacillus strain can cleavage protein specifically at -Lys. Peptides PVNKP (SEQ ID NO. 7), GYPVV (SEQ ID NO. 18), ELSEQDIFV (SEQ ID NO. 19), ATSNLNF (SEQ ID NO. 20), PIPFP (SEQ ID NO. 10), LKPDNR (SEQ ID NO. 9) and IPRPRPRP (SEQ ID NO. 8) were identified from CKPNO01. Since ACE prefers substrates containing a hydrophobic (aromatic or branched side-chains) amino acid residue at the C-terminal position, these peptides may have the potential of ACE inhibition activity, which was then confirmed by testing with individual synthesized peptides, see Table 6.

### Measurement of ACE Inhibitory Activity

Angiotensin-I-converting enzyme inhibitory activity was determined using FAPGG as the substrate [11]. A mixture containing 100 μl of 1 mM FAPGG, 20 μl of the soy peptide solution, and 80 μl of reaction solution (100 mM Tris-HCl, pH 8.3, containing 0.3 M NaCl, 10 mM ZnCl2, and 0.025 U/ml rabbit lung angiotensin converting enzyme (Sigma Chemical Company, St. Louis, Mo.)) was added to a 96-well plate. The reaction was carried out at 37° C. for 30 minutes and the absorbance of the mixture was read using a spectrophotometry set at 340 nm to determine the enzyme activity. 20 μl of distilled water was used in the place of the soy peptide solution in each assay as a negative control. The inhibitory potency (IC50) of each fraction of the soy peptides was determined from plots of percentage inhibition vs. log 10[soy peptide] (mg/L). IC50 value of Angiotensin-I-converting enzyme, defined as the amount of soy peptide that produced a 50% inhibition of ACE activity, was determined for selected fractions. Captopril was used as the positive control.

### Effect of Soy Peptides on Systolic Blood Pressure in Spontaneous Hypertensive Rats (SHR)

### Continuous Administration of Soy Peptides to SHR

Eight-week old spontaneously hypertensive rats (SHR) were purchased from Beijing Vitalriver Co. The rats were kept separately in movable stainless rack in the environment of 25±3° C., relative humidity of 50-70%, and 12 hours light/dark cycle. The blood pressure of the rats was measured using the non-invasive automatic sphygmomanometer (Power Lab ML125).

TABLE 1
Various treatments using CKPN001 in the SHR animal study

| | Dosage | Number |
|---|---|---|
| Treatment | (mg/kg BW/day) | of rats |
| Control | 0 | 10 |
| Soy peptides (Low) | 10 | 10 |
| Soy peptides (Medium) | 30 | 10 |
| Soy peptides (High) | 90 | 10 |

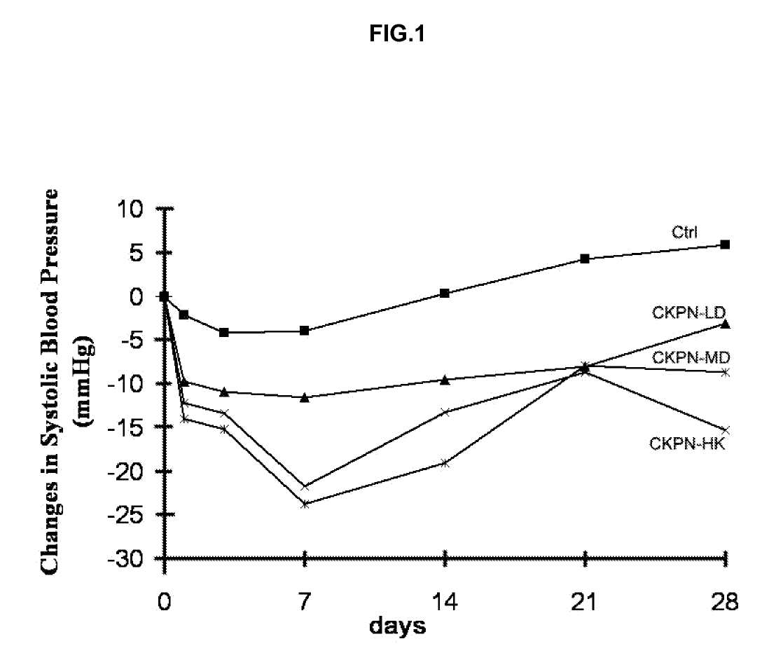

As shown in Table 1, the rats were divided into four groups, including one negative control and three test groups. Three dosages of CKPN001 produced as described in the foregoing were fed into three treated groups of the rats. As shown in Tables 2 and 3 as well as FIG. 1, the soy peptides were dissolved in a solution of 0.9% NaCl and then orally fed into the rats. The control group received only the saline. The blood pressure of the rats was measured 1, 3, 7, 14, and 21 days after the oral administration. The average value of systolic blood pressures for each treated group was compared with that of the control group. The statistical software SPSS 13.0 was used for data analysis.

Seven days after administration, the SHRs which received 30 and 90 mg/kg/day of soy peptides exhibited significantly lower blood pressure (p<0.01). The SHRs which received 10 mg/kg/day of soy peptides also exhibited decreased blood pressure, but not statistically significant. Furthermore, the decrease in blood pressure is also obvious when the SHRs received soy peptides for 14, 21, and 28 days as compared to the control group. Therefore, continuous administration of the soy peptides prepared in the present invention at 30 and 90 mg/kg/day had a hypotensive effect on SHRs.

TABLE 2
Hypotensive effect of the soy peptides on SHR (continuous administration)

| | Systolic Blood Pressure (mmHg) | | | | | | |
|---|---|---|---|---|---|---|---|
| Soy | Days after administration | | | | | | |
| Peptides | 0 | 1 | 3 | 7 | 14 | 21 | 28 |
| 0 mg/kg | 184.8 ± 10.3 | 182.6 ± 10.5 | 181.2 ± 13.5 | 181.4 ± 14.0 | 183.6 ± 15.0 | 189.6 ± 12.0 | 191.3 ± 7.9 |
| 10 mg/kg | 182.1 ± 13.7 | 172.3 ± 15.5 | 171.1 ± 13.6 | 170.5 ± 16.4 | 172.5 ± 16.1 | 174.0 ± 12.5 | 178.3 ± 13.1 |
| 30 mg/kg | 182.0 ± 12.5 | 169.8 ± 17.8 | 168.6 ± 14.3 | 160.3 ± 15.6** | 168.7 ± 14.7* | 173.3 ± 11.9 | 168.7 ± 15.1* |
| 90 mg/kg | 183.8 ± 18.9 | 170.0 ± 17.7 | 168.6 ± 14.5 | 160.0 ± 12.0** | 164.7 ± 12.9** | 175.8 ± 10.0* | 176.4 ± 16.1 |

*p < 0.05,
**p < 0.01 vs control group

TABLE 3
Changes in systolic blood pressure after administration of soy peptides
(continuous administration)

| | Changes in Systolic Blood Pressure (mmHg) | | | | | | |
|---|---|---|---|---|---|---|---|
| Soy | Days after administration | | | | | | |
| Peptides | 0 | 1 | 3 | 7 | 14 | 21 | 28 |
| 0 mg/kg | 0.00 ± 0.00 | −2.18 ± 9.88 | −4.20 ± 14.23 | −4.03 ± 16.86 | 0.23 ± 18.01 | 4.20 ± 14.82 | 5.87 ± 10.37 |
| 10 mg/kg | 0.00 ± 0.00 | −9.82 ± 3.61 | −11.00 ± 10.42 | −11.56 ± 17.12 | −9.61 ± 14.47 | −8.04 ± 15.22 | −3.14 ± 21.27 |
| 30 mg/kg | 0.00 ± 0.00 | −12.20 ± 8.27** | −13.40 ± 14.75 | −21.73 ± 23.78 | −13.33 ± 17.42 | −8.7 ± 17.62 | −15.33 ± 21.91** |
| 90 mg/kg | 0.00 ± 0.00 | −14.07 ± 11.53** | −15.23 ± 11.13** | −23.83 ± 14.68* | −19.09 ± 24.22 | −8.02 ± 23.27 | −8.71 ± 21.99** |

*p < 0.05,
**p < 0.01 vs control group

### Single Dose Administration of Soy Peptides to SHR

Thirteen-week old male SHRs (body weight, 180 to 230 g) were divided into four groups including one negative control and three test groups, with each containing 8 rats. Systolic blood pressure (SAP), diastolic blood pressure (DAP) and mean arterial pressure (MAP) were recorded from left carotid artery of the rats which was anesthetized with urethane. For the treated groups, the soy peptides were dissolved in water and administrated at a rate of 10, 30, or 90 mg/kg. For the control group, the same volume of saline was orally administrated. The blood pressure of the rats was measured 30, 60, 90, 120, 150, and 180 mins after administration.

As shown in Tables 3 and 4, a decrease in blood pressure was observed in the treated groups 60 mins after administration. The reduction in blood pressure was most evident at 120-150 mins after administration of soy peptides. At 90 mins after administration of soy peptides of 10, 30 and 90 mg/kg body weight, the mean arterial pressure was decreased 14.5, 16.3 and 14.0 mmHg, respectively, as compared to pre-drug administration (p<0.05). Results indicated that single dose administration of the soy peptides prepared in the present invention had a hypotensive effect on SHRs.

TABLE 4
Hypotensive effect of soy peptides on SHR (single-dose administration)

| | Mean Arterial Pressure (mmHg) | | | | | | |
|---|---|---|---|---|---|---|---|
| Soy | Minutes after administration | | | | | | |
| Peptides | 0 | 30 | 60 | 90 | 120 | 150 | 180 |
| 0 mg/kg | 141 ± 19 | 139 ± 20 | 137 ± 19 | 137 ± 17 | 137 ± 17 | 137 ± 20 | 135 ± 19 |
| 10 mg/kg | 157 ± 14 | 150 ± 17 | 148 ± 16 | 143 ± 17 | 142 ± 18 | 143 ± 18 | 146 ± 18 |
| 30 mg/kg | 148 ± 15 | 142 ± 16 | 139 ± 15 | 132 ± 17 | 128 ± 16* | 127 ± 18* | 132 ± 17 |
| 90 mg/kg | 153 ± 12 | 148 ± 10 | 145 ± 13 | 139 ± 13* | 136 ± 12* | 137 ± 12* | 139 ± 12* |

*p < 0.05 vs before administration

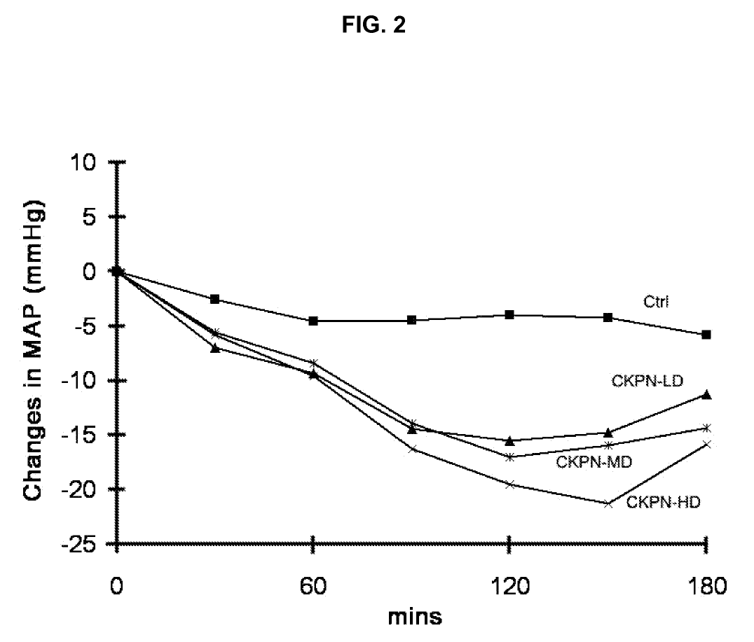

The results shown in Table 5 and FIG. 2, indicates that single dose administration of the soy peptides prepared in the present invention had a hypotensive effect SHRs as reflect on mean arterial pressure (MAP).

TABLE 5
Changes in mean arterial pressure after administration of soy peptides (single-dose administration)

| | Changes in Mean Arterial Pressure (mmHg) | | | | | | |
|---|---|---|---|---|---|---|---|
| Soy | Minutes after administration | | | | | | |
| Peptides | 0 | 30 | 60 | 90 | 120 | 150 | 180 |
| 0 mg/kg | 0 | −2.6 ± 3.2 | −4.6 ± 2.2 | −4.5 ± 4.2 | −4.0 ± 5.4 | −4.3 ± 4.0 | −5.9 ± 4.8 |
| 10 mg/kg | 0 | −70 ± 4.4* | −9.4 ± 5.9 | −14.5 ± 7.5** | −15.6 ± 6.2** | −14.8 ± 7.8** | −11.3 ± 7.3 |
| 30 mg/kg | 0 | −5.9 ± 4.5 | −9.6 ± 4.2** | −16.3 ± 6.5** | −19.6 ± 7.3** | −21.3 ± 8.5** | −15.9 ± 9.3* |
| 90 mg/kg | 0 | −5.6 ± 8.4 | −8.5 ± 4.3* | −14.0 ± 5.3** | −17.1 ± 7.9** | −16.0 ± 7.3** | −14.4 ± 6.3* |

*p < 0.05,
**p < 0.01 vs before administration

TABLE 6

The ACE inhibitory activity
of synthetic peptides

| | | |
|---|---|---|
| Peptide Name | Sequence | ACE Inhibition (IC₅₀, mM) |

| | | |
|---|---|---|
| IP-8 | IPRPRPRP | 0.0072 ± 0.0017 |
| PP-5 | PIPFP | 0.0660 ± 0.0153 |
| PP-5-1 | PVNKP | 0.0461 ± 0.0052 |
| LR-6 | LKPDNR | 0.1208 ± 0.0017 |

The peptides in Table 6 were identified through MALDI TOF/TOF analyzer, and were chemical synthesized by solid phase synthesis and confirmed by HPLC and mass spectrometry analysis. They were then tested for their ACE inhibitory activities. IC₅₀ values were calculated and listed in the table.

PP-5, PP-5-1, and LR-6 are believed to be novel peptides. While IP-8 was reported previously for another indication, it is believed to be novel to use it for anti-hypertensive purpose.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

### REFERENCES

1. Lazartigues E, Feng Y, and Lavoie J L. (2007) The two fACEs of the tissue renin-angiotensin systems: implication in cardiovascular diseases. Curr Pharm Des 13:1231-45
2. Petrillo E W, Jr., and Ondetti M A. (1982) Angiotensin converting enzyme inhibitors: medicinal chemistry and biological actions. Med Res Rev 2:1-41.
3. Chow C M. (1999) Evaluation and treatment of hypertension. Rheum Dis Clin North Am 25:521-37.
4. Kitts D D, and Weiler K. (2003) Bioactive proteins and peptides from food sources. Applications of bioprocesses used in isolation and recovery. Curr Pharm Des 9: 1309-23.
5. Korhonen H, and Pihlanto A. (2003) Food-derived bioactive peptides-opportunities for designing future foods. Curr Pharm Des. 9: 1297-308.
6. Tsumura K, Saito T, Kugimiya W, and Inouye K. (2004) Selective proteolysis of the glycinin and β-conglycinin fractions in a soy protein isolate by pepsin and papain with controlled pH and temperature. J Food Sci 69: 363-367.
7. Chen J, Okada, T, Muramoto K, Suetsuna K, Yang S. (2003) identification of angiotensin I-converting enzyme inhibitory peptides derived from the peptic digest of soybean protein. J Food Biochem 26: 543-54.
8. Chen J R, Yang S C, Suetsuna K, Chao J C J. (2004) Soybean protein-derived hydrolysate affects blood pressure in spontaneously hypertensive rats. J Food Biochem 28: 61-73.
9. Wu J and Ding X. (2001) Hypotensive and physiological effect of angiotensin converting enzyme inhibitory peptides derived from soy protein on spontaneously hypertensive rats. J Agric Food Chem 49: 501-6.
10. Rick W. (1974) in Methods of Enzymatic Analysis (Bergmeyer, H. U. ed) 2^{nd} ed., Volume II, pp1021-24, Academic Press, Inc., New York, N.Y.
11. Murray B A, Walsh D J, and FitzGerald R J. (2004). Modification of the furanacryloyl-L-phenylalanylglycylglycine assay for determination of angiotensin-I-converting enzyme inhibitory activity. J Biochem Biophys Methods 59: 127-37.

What is claimed is:

1. A method of making a soy peptide composition, comprising steps of:
(a) isolating a Bacillus species intrinsic to soybean food which produces a proteases;
(b) inoculating said Bacillus species in a solution of soy protein to start a fermentation process for decomposing said soy protein;
(c) isolating peptides with a molecular weight equal to or less than 10 kD from said solution of soy protein that has undergone said fermentation process of step (b); and
(d) drying said peptides to afford a soy peptide composition, which possesses an activity of lowering blood pressure when administered to people.

2. The method of claim 1, wherein said soybean food is natto, douchi, furu, ground bean sauce, or bean paste.

3. The method of claim 2, wherein said fermentation process of step (b) was conducted in the presence of D-glucose.

4. The method of claim 3, wherein said isolating step (c) was achieved through centrifugation and ultra-filtration.

5. A soy peptide composition, being produced according to the method of claim 1.

6. The soy peptide composition of claim 5, being indicated to be useful for lowering blood pressure.

7. The soy peptide composition of claim 5, possessing an activity of lowering blood pressure and comprising one or more peptides derived from the alpha subunit of beta conglycinin region 87-102.

8. The soy peptide composition of claim 7, wherein said one or more peptides are selected from the group consisting of PFPRPPHQKEE (SEQ ID NO. 1), DERPFPFPRPPHQK (SEQ ID NO. 2), ERQFPFPRPPHQK (SEQ ID NO. 3), RQFPFPRPPHQK (SEQ ID NO. 4), QFPFPRPPHQK (SEQ ID NO. 5) and ERQFPFPRPPH(SEQ ID NO. 6).

9. The soy peptide composition of claim 5, possessing an activity of lowering blood pressure and comprising one or more peptides selected from the group consisting of PVNKP (SEQ ID NO. 7), IPRPRPRP (SEQ ID NO. 8), LKPDNR (SEQ ID NO. 9) and PIPFP (SEQ ID NO. 10).

10. The soy peptide composition of claim 9, comprising PVNKP (SEQ ID NO. 7).

11. The soy peptide composition of claim 9, comprising IPRPRPRP (SEQ ID NO. 8).

12. The soy peptide composition of claim 9, comprising LKPDNR(SEQ ID NO. 9).

13. The soy peptide composition of claim 9, comprising PIPFP (SEQ ID NO. 10).

14. The soy peptide composition of claim 9, comprising PFPRPPHQKEE (SEQ ID NO. 1).

15. The soy peptide composition of claim 9, comprising DERPFPFPRPPHQK (SEQ ID NO. 2).

16. The soy peptide composition of claim 9, comprising ERQFPFPRPPHQK (SEQ ID NO. 3).

17. The soy peptide composition of claim 9, comprising RQFPFPRPPHQK (SEQ ID NO. 4).

18. The soy peptide composition of claim 9, comprising QFPFPRPPHQK (SEQ ID NO. 5).

19. The soy peptide composition of claim 9, comprising ERQFPFPRPPH (SEQ ID NO. 6).


SEQUENCE LISTING (20 sequences)

SEQ ID NO: 1 - protein, 11 aa, soy
PFPRPPHQKEE

SEQ ID NO: 2 - protein, 14 aa, soy
DERPFPFPRPPHQK

SEQ ID NO: 3 - protein, 13 aa, soy
ERQFPFPRPPHQK

SEQ ID NO: 4 - protein, 12 aa, soy
RQFPFPRPPHQK

SEQ ID NO: 5 - protein, 11 aa, soy
QFPFPRPPHQK

SEQ ID NO: 6 - protein, 11 aa, soy
ERQFPFPRPPH

SEQ ID NO: 7 - protein, 5 aa, soy
PVNKP

SEQ ID NO: 8 - protein, 8 aa, soy
IPRPRPRP

SEQ ID NO: 9 - protein, 6 aa, soy
LKPDNR

SEQ ID NO: 10 - protein, 5 aa, soy
PIPFP

SEQ ID NO: 11 - protein, 6 aa, soy
YLAGNQ

SEQ ID NO: 12 - protein, 4 aa, soy
IYLL

SEQ ID NO: 13 - protein, 5 aa, soy
YVVFK

SEQ ID NO: 14 - protein, 7 aa, soy
PNNKPFQ

SEQ ID NO: 15 - protein, 6 aa, soy
NWGPLV

SEQ ID NO: 16 - protein, 9 aa, soy
IPPGVPYWT

SEQ ID NO: 17 - protein, 5 aa, soy
TPRVF

SEQ ID NO: 18 - protein, 5 aa, soy
GYPVV

SEQ ID NO: 19 - protein, 9 aa, soy
ELSEQDIFV

SEQ ID NO: 20 - protein, 7 aa, soy
ATSNLNF